(19) United States
(12) Patent Application Publication
(10) Pub. No.: US 2011/0281858 A1
(43) Pub. Date: Nov. 17, 2011

(54) AEROSOL FORMULATION FOR THE INHALATION OF BETA AGONISTS

(75) Inventor: Michael Aven, Mainz (DE)

(73) Assignee: BOEHRINGER INGELHEIM INTERNATIONAL GMBH, Ingelheim am Rhein (DE)

(21) Appl. No.: 13/129,955

(22) PCT Filed: Nov. 18, 2009
(86) PCT No.: PCT/EP2009/065407

§ 371 Date: Jul. 13, 2011

(30) Foreign Application Priority Data
Nov. 21, 2008 (EP) 08169649.4

(51) Int. Cl. A61K 31/538; A61M 11/00; A61P 11/00

(52) U.S. Cl. 514/230.5; 128/200.14

(57) ABSTRACT

The present invention relates to a propellant-free aerosol formulation which [contains] one or more compounds of general formula (1) wherein the groups R¹, R², R³ and X⁻ may have the meanings given in the claims and in the description, for inhalation.

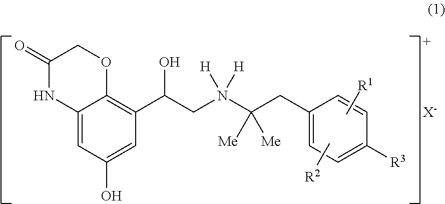

The present invention relates to a propellant-free aerosol formulation which contains one or more compounds of general formula 1

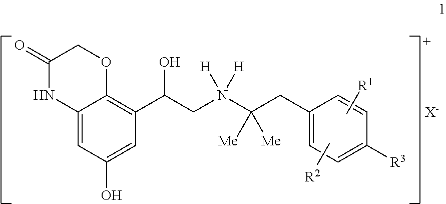

wherein the groups R¹, R², R³ and X⁻ may have the meanings given in the claims and in the description, for inhalation.

### BACKGROUND TO THE INVENTION

Betamimetics (β-adrenergic substances) are known from the prior art. For example reference may be made in this respect to the disclosures of EP 1562603 or U.S. Pat. No. 7,056,916 which propose betamimetics for treating a variety of diseases. For the drug therapy of diseases it is often desirable to prepare medicaments with a longer duration of activity. As a rule, this ensures that the concentration of the active substance in the body needed to achieve the therapeutic effect is guaranteed for a longer period without the need to re-administer the drug at frequent intervals. Moreover, giving an active substance at longer time intervals contributes to the wellbeing of the patient to a high degree. It is particularly desirable to prepare a pharmaceutical composition which can be used therapeutically by administration once a day (single dose). The use of a drug once a day has the advantage that the patient can become accustomed relatively quickly to regularly taking the drug at certain times of the day.

The aim of the present invention is therefore to provide medicament formulations for inhalation which on the one hand have a therapeutic benefit for example in the treatment of respiratory complaints and are also characterised by a longer duration of activity and can thus be used to prepare medicaments with a longer duration of activity.

### DESCRIPTION OF THE INVENTION

To solve the problems stated above, the present invention proposes the following medicament formulations. The medicament formulations according to the invention are propellant-free medicament formulations containing
as sole active substance one or more compounds of general formula 1

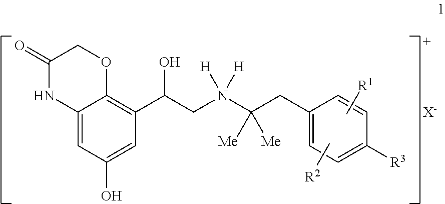

wherein
R¹ denotes hydrogen, fluorine, chlorine or methyl, preferably hydrogen;
R² denotes hydrogen, fluorine, chlorine or methyl, preferably hydrogen;
R³ denotes methyl, methoxy, ethoxy, fluorine, chlorine, bromine, O—CH₂—COOH or O—CH₂—COOethyl;
X⁻ denotes an anion with a single negative charge selected from among chloride, bromide, sulphate, methanesulphonate, maleate, acetate, benzoate, citrate, salicylate, trifluoroacetate, fumarate, tartrate and succinate;
optionally in the form of the tautomers, enantiomers, mixtures of enantiomers, racemates or solvates thereof;
benzalkonium chloride;
at least one pharmacologically acceptable acid,
and, as solvent, water, ethanol or a mixture of water and ethanol,
optionally other pharmacologically acceptable excipients and/or complexing agents characterised in that the content of compound 1 is 55 to 200 μmol per 100 ml solution.

Also preferred are medicament formulations which contain compounds of general formula 1, wherein
R³ denotes methoxy, ethoxy, fluorine, O—CH₂—COOH, O—CH₂—COOmethyl or O—CH₂—COOethyl;
   and R¹, R² and X⁻ may have the meanings given above, optionally in the form of the tautomers, enantiomers, mixtures of enantiomers, racemates or solvates thereof.

Also preferred are medicament formulations which contain compounds of general formula 1, wherein
R¹ and R² denote hydrogen or fluorine;
R³ denotes fluorine, methoxy, ethoxy, O—CH₂—COOH, preferably fluorine, methoxy or ethoxy;
   and X⁻ may have one of the meanings given above, optionally in the form of the tautomers, enantiomers, mixtures of enantiomers, racemates or solvates thereof.

Also preferred are medicament formulations which contain compounds of general formula 1, which are selected from among the group consisting of:
6-hydroxy-8-{1-hydroxy-2-[2-(4-methoxy-phenyl)-1,1-dimethyl-ethylamino]-ethyl}-4H-benzo[1,4]oxazin-3-one;
6-hydroxy-8-{1-hydroxy-2-[2-(ethyl 4-phenoxy-acetate)-1,1-dimethyl-ethylamino]-ethyl}-4H-benzo[1,4]oxazin-3-one;
6-hydroxy-8-{1-hydroxy-2-[2-(4-phenoxy-acetic acid)-1,1-dimethyl-ethylamino]-ethyl}-4H-benzo[1,4]oxazin-3-one;
8-{2-[2-(4-fluoro-3-methyl-phenyl)-1,1-dimethyl-ethylamino]-1-hydroxy-ethyl}-6-hydroxy-4H-benzo[1,4]oxazin-3-one;
8-{2-[2-(4-fluoro-2-methyl-phenyl)-1,1-dimethyl-ethylamino]-1-hydroxy-ethyl}-6-hydroxy-4H-benzo[1,4]oxazin-3-one;
8-{2-[2-(2,4-difluoro-phenyl)-1,1-dimethyl-ethylamino]-1-hydroxy-ethyl}-6-hydroxy-4H-benzo[1,4]oxazin-3-one;
8-{2-[2-(3,5-difluoro-phenyl)-1,1-dimethyl-ethylamino]-1-hydroxy-ethyl}-6-hydroxy-4H-benzo[1,4]oxazin-3-one;
8-{2-[2-(4-ethoxy-phenyl)-1,1-dimethyl-ethylamino]-1-hydroxy-ethyl}-6-hydroxy-4H-benzo[1,4]oxazin-3-one;
8-{2-[2-(3,5-dimethyl-phenyl)-1,1-dimethyl-ethylamino]-1-hydroxy-ethyl}-6-hydroxy-4H-benzo[1,4]oxazin-3-one;
8-{2-[2-(3,4-difluoro-phenyl)-1,1-dimethyl-ethylamino]-1-hydroxy-ethyl}-6-hydroxy-4H-benzo[1,4]oxazin-3-one;
8-{2-[2-(2-chloro-4-fluoro-phenyl)-1,1-dimethyl-ethylamino]-1-hydroxy-ethyl}-6-hydroxy-4H-benzo[1,4]oxazin-3-one;
8-{2-[2-(4-chloro-phenyl)-1,1-dimethyl-ethylamino]-1-hydroxy-ethyl}-6-hydroxy-4H-benzo[1,4]oxazin-3-one;
8-{2-[2-(4-bromo-phenyl)-1,1-dimethyl-ethylamino]-1-hydroxy-ethyl}-6-hydroxy-4H-benzo[1,4]oxazin-3-one;
8-{2-[2-(4-fluoro-phenyl)-1,1-dimethyl-ethylamino]-1-hydroxy-ethyl}-6-hydroxy-4H-benzo[1,4]oxazin-3-one;
8-{2-[2-(4-fluoro-3-methoxy-phenyl)-1,1-dimethyl-ethylamino]-1-hydroxy-ethyl}-6-hydroxy-4H-benzo[1,4]oxazin-3-one;
8-{2-[2-(4-fluoro-2,6-dimethyl-phenyl)-1,1-dimethyl-ethylamino]-1-hydroxy-ethyl}-6-hydroxy-4H-benzo[1,4]oxazin-3-one;
8-{2-[2-(4-chloro-2-methyl-phenyl)-1,1-dimethyl-ethylamino]-1-hydroxy-ethyl}-6-hydroxy-4H-benzo[1,4]oxazin-3-one;
8-{2-[2-(4-chloro-3-fluoro-phenyl)-1,1-dimethyl-ethylamino]-1-hydroxy-ethyl}-6-hydroxy-4H-benzo[1,4]oxazin-3-one;
8-{2-[2-(4-chloro-2-fluoro-phenyl)-1,1-dimethyl-ethylamino]-1-hydroxy-ethyl}-6-hydroxy-4H-benzo[1,4]oxazin-3-one;
8-{2-[2-(3-chloro-4-fluoro-phenyl)-1,1-dimethyl-ethylamino]-1-hydroxy-ethyl}-6-hydroxy-4H-benzo[1,4]oxazin-3-one;
8-{2-[2-(2,6-difluoro-4-methoxy-phenyl)-1,1-dimethyl-ethylamino]-1-hydroxy-ethyl}-6-hydroxy-4H-benzo[1,4]oxazin-3-one;
8-{2-[2-(2,5-difluoro-4-methoxy-phenyl)-1,1-dimethyl-ethylamino]-1-hydroxy-ethyl}-6-hydroxy-4H-benzo[1,4]oxazin-3-one;
8-{2-[2-(4-fluoro-3,5-dimethyl-phenyl)-1,1-dimethyl-ethylamino]-1-hydroxy-ethyl}-6-hydroxy-4H-benzo[1,4]oxazin-3-one;
8-{2-[2-(3,5-dichloro-phenyl)-1,1-dimethyl-ethylamino]-1-hydroxy-ethyl}-6-hydroxy-4H-benzo[1,4]oxazin-3-one;
8-{2-[2-(4-chloro-3-methyl-phenyl)-1,1-dimethyl-ethylamino]-1-hydroxy-ethyl}-6-hydroxy-4H-benzo[1,4]oxazin-3-one;
8-{2-[2-(3,4,5-trifluoro-phenyl)-1,1-dimethyl-ethylamino]-1-hydroxy-ethyl}-6-hydroxy-4H-benzo[1,4]oxazin-3-one;
8-{2-[2-(3-methyl-phenyl)-1,1-dimethyl-ethylamino]-1-hydroxy-ethyl}-6-hydroxy-4H-benzo[1,4]oxazin-3-one and
8-{2-[2-(3,4-dichloro-phenyl)-1,1-dimethyl-ethylamino]-1-hydroxy-ethyl}-6-hydroxy-4H-benzo[1,4]oxazin-3-one,
   in each case in the form of an acid addition salt with an acid HX, wherein X⁻ may have one of the meanings given above, and optionally in the form of the tautomers, enantiomers, mixtures of enantiomers, racemates or solvates thereof.

Also particularly preferred are medicament formulations which contain compounds of formulae 1a-1e:

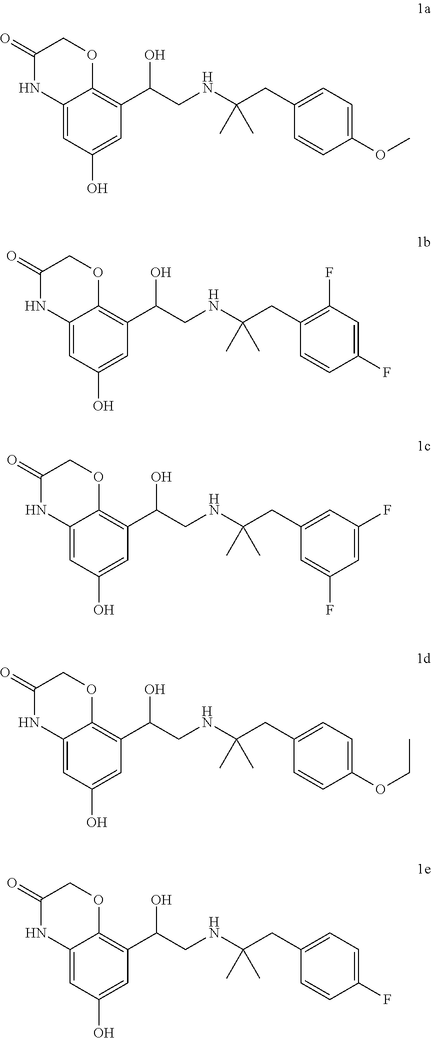

in each case in the form of an acid addition salt with an acid HX, wherein X⁻ may have one of the meanings given above, and optionally in the form of the tautomers, enantiomers, mixtures of enantiomers, racemates or solvates thereof.

### DETAILED DESCRIPTION OF THE INVENTION

The medicament formulations according to the invention contain as solvent pure water, pure ethanol or mixtures of ethanol and water. If ethanol-water mixtures are used, the percentage amount of ethanol by mass in these mixtures is preferably in the range between 5 and 99% ethanol, particularly preferably in the range from 10 to 96% ethanol. Most particularly preferred medicament formulations for the purposes of the present invention contain as solvent pure water, pure ethanol or ethanol-water mixtures containing between 50 and 92%, particularly preferably between 69 and 91% ethanol. If desired, other co-solvents may be used besides ethanol and water. According to the invention, however, it is preferable not to use an additional solvent.

The compounds of formula 1 may optionally be present in the medicament formulations according to the invention in the form of their tautomers. By tautomerism is meant the occurrence of isomeric compounds which are formed by displacing σ- or π-bonds and which may be present in equilibrium. Examples of possible tautomeric forms of the compounds of formula 1 are

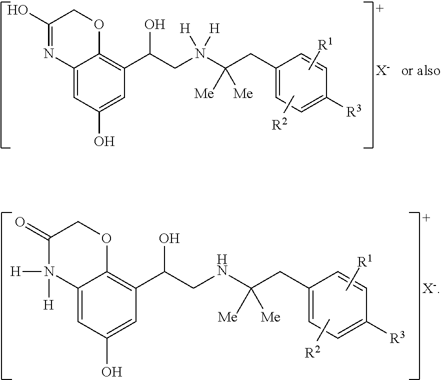

In another aspect the present invention relates to medicament formulations that contain the above-mentioned compounds of formula 1 in the form of the individual optical isomers, mixtures of the individual enantiomers or racemates. Particularly preferred are medicament formulations which contain the above-mentioned compounds of formula 1 in the form of the compounds with high enantiomeric purity, while the R-enantiomers of the compounds of formula 1 are of exceptional importance according to the invention. These R-enantiomers may be represented by general formula R-1

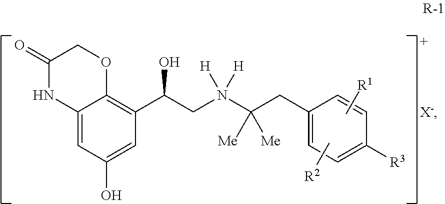

wherein the groups R¹, R², R³ and X⁻ may have the meanings given above.

In another aspect the present invention relates to the use of the medicament formulations according to the invention for preparing a pharmaceutical composition for the treatment of respiratory complaints, which are selected from among obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the compounds are used as described above to prepare a pharmaceutical composition for the treatment of obstructive pulmonary diseases selected from among bronchial asthma, paediatric asthma, severe asthma, acute asthma attacks, chronic bronchitis and chronic obstructive pulmonary disease (COPD), while it is particularly preferable according to the invention to use them for preparing a pharmaceutical composition for the treatment of bronchial asthma or COPD.

It is also preferable to use the medicament formulations according to the invention to prepare a medicament for the treatment of pulmonary emphysema which has its origins in COPD (chronic obstructive pulmonary disease) or α1-proteinase inhibitor deficiency.

It is also preferable to use the medicament formulations according to the invention to prepare a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament formulations according to the invention to prepare a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic scleroderma or sarcoidosis, granulomatoses, such as for example Boeck's disease, idiopathic interstitial pneumonia or idiopathic pulmonary fibrosis (IPF).

It is also preferable to use the medicament formulations according to the invention to prepare a pharmaceutical composition for the treatment of cystic fibrosis or mucoviscidosis.

It is also preferable to use the medicament formulations according to the invention to prepare a pharmaceutical composition for the treatment of bronchitis, such as for example bronchitis caused by bacterial or viral infection, allergic bronchitis and toxic bronchitis.

It is also preferable to use the medicament formulations according to the invention to prepare a pharmaceutical composition for the treatment of bronchiectasis.

It is also preferable to use the medicament formulations according to the invention to prepare a pharmaceutical composition for the treatment of ARDS (adult respiratory distress syndrome).

It is also preferable to use the medicament formulations according to the invention to prepare a pharmaceutical composition for the treatment of pulmonary oedema, for example toxic pulmonary oedema after aspiration or inhalation of toxic substances and foreign substances.

Particularly preferably, the present invention relates to the use of the medicament formulations according to the invention for preparing a pharmaceutical composition for the treatment of asthma or COPD. Also of particular importance is the above-mentioned use for preparing a pharmaceutical composition for once-a-day treatment of inflammatory and obstructive respiratory complaints, particularly for the once-a-day treatment of asthma or COPD.

Moreover the present invention relates to a process for the treatment of the above-mentioned diseases, characterised in that one or more of the above-mentioned medicament formulations according to the invention are administered in therapeutically effective amounts.

The present invention relates to liquid active substance formulations of these compounds which can be administered by inhalation; the liquid formulations according to the invention have to meet high quality standards. The formulations according to the invention may be inhaled by oral or nasal route. To achieve an optimum distribution of the active substances in the lung it makes sense to use a liquid formulation without propellant gases administered using suitable inhalers. A formulation of this kind may be inhaled both by oral route and by nasal route. Those inhalers which are capable of nebulising a small amount of a liquid formulation in the dosage needed for therapeutic purposes within a few seconds into an aerosol suitable for therapeutic inhalation are particularly suitable. Within the scope of the invention, preferred nebulisers are those in which an amount of less than 100 microlitres, preferably less than 50 microlitres, most preferably less than 25 microlitres of active substance solution can be nebulised preferably in one puff or two puffs to form an aerosol having an average particle size (or particle diameter) of less than 20 microns, preferably less than 10 microns, so that the inhalable part of the aerosol already corresponds to the therapeutically effective quantity.

An apparatus of this kind for the propellant-free administration of a metered amount of a liquid pharmaceutical composition for inhalation is described in detail for example in International Patent Application WO 91/14468 “Atomizing Device and Methods” and also in WO 97/12687, cf. FIGS. 6a and 6b and the accompanying description. In a nebuliser of this kind a pharmaceutical solution is converted by means of a high pressure of up to 500 bar into an aerosol destined for the lungs, which is sprayed. Within the scope of the present specification reference is expressly made to the entire contents of the literature mentioned above.

In inhalers of this kind the formulations of solutions are stored in a reservoir. It is essential that the active substance formulations used are sufficiently stable when stored and at the same time are such that they can be administered directly, if possible without any further handling, in accordance with their medical purpose. Moreover, they must not contain any ingredients which might interact with the inhaler in such a way as to damage the inhaler or the pharmaceutical quality of the solution or of the aerosol produced.

To nebulise the solution a special nozzle is used as described for example in WO 94/07607 or WO 99/16530. Reference is expressly made here to both these publications.

The aim of the invention is to provide an aqueous, ethanolic or aqueous-ethanolic formulation of the compound of formula 1 which meets the high standards required to ensure optimum nebulisation of a solution using the inhalers mentioned above. The active substance formulations according to the invention must be of sufficiently high pharmaceutical quality, i.e. they should be pharmaceutically stable over a storage time of some years, preferably at least one year, more preferably two years. These propellant-free formulations of solutions must also be capable of being nebulised by means of an inhaler under pressure, while the composition delivered in the aerosol produced is within a specified range.

Within the scope of the present invention It is particularly preferable to use those compounds of formula 1 wherein X⁻ is selected from among chloride, maleate, salicylate, fumarate or succinate, optionally in the form of the hydrates and solvates thereof.

Particularly preferred within the scope of the present invention are those formulations that contain the compound of formula 1 wherein X⁻ denotes chloride.

References to the compound of formula 1 always include within the scope of the present invention all the possible amorphous and crystalline modifications of this compound. References to the compound of formula 1 also include within the scope of the present invention all the possible solvates and hydrates which may be formed from this compound.

Any reference made to the compound 1′ within the scope of the present invention is to be regarded as a reference to the pharmacologically active free base of the following formula contained in the salts 1:

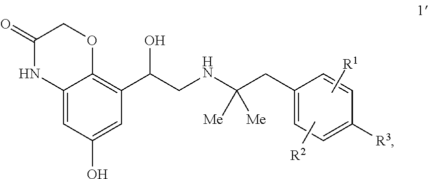

wherein the groups R¹, R², R³ and X⁻ may have the meanings given above.

In another aspect the present invention relates to medicament formulations containing as sole active substance a free base of formula 1′ wherein the groups R¹, R², R³ and X⁻ may have the meanings given above, optionally in the form of the tautomers, enantiomers, mixtures of the enantiomers, racemates or solvates thereof, at least one pharmacologically acceptable acid, optionally further pharmacologically acceptable excipients and/or complexing agents, as well as water, ethanol or a mixture of water and ethanol as solvent.

According to the invention, the formulation preferably contains only one compound of formula 1. However, the formulation may also contain a mixture of different salts of formula 1. If the medicament formulations according to the invention contain different salts of formula 1 the preferred formulations according to the invention are those wherein the various salts represent different salts of the same free base of formula 1′. Formulations which contain different active substances from those of formula 1 are not a subject of the invention.

The concentration of the compound of formula 1 based on the amount of pharmacologically active free base 1′ in the medicament formulation according to the invention is about 55 to 200 μmol per 100 ml, preferably about 56 to 150 μmol per 100 ml, particularly preferably 57 to 135 μmol per 100 ml according to the invention. Particularly preferably 100 ml of the formulations according to the invention contain about 58 to about 120 μmol of 1′.

The pH of the formulation according to the invention is preferably in the range from 2.0 and 6.5, preferably between 2.2 and 5.0, particularly preferably between about 3.0 and 4.5 according to the invention.

The pH is adjusted by the addition of pharmacologically acceptable acids. Pharmacologically acceptable inorganic acids or organic acids may be used for this purpose. Examples of preferred inorganic acids are selected from the group consisting of hydrochloric acid, hydrobromic acid, nitric acid, sulphuric acid and phosphoric acid.

Examples of particularly suitable organic acids are selected from the group consisting of ascorbic acid, citric acid, malic acid, tartaric acid, maleic acid, succinic acid, fumaric acid, acetic acid, formic acid and propionic acid. Preferred inorganic acids are hydrochloric acid and sulphuric acid, of which hydrochloric acid is particularly important according to the invention. Of the organic acids, ascorbic acid, fumaric acid and citric acid are preferred, of which citric acid is particularly preferred according to the invention. If desired, mixtures of the abovementioned acids may also be used, particularly in the case of acids which have other properties in addition to their acidifying properties, e.g. those which act as flavourings or antioxidants, such as for example citric acid or ascorbic acid. If desired, pharmacologically acceptable bases may also be used to titrate the pH precisely. Suitable bases include for example alkali metal hydroxides and alkali metal carbonates. The preferred alkali metal ion is sodium. If bases of this kind are used, care must be taken to ensure that the resulting salts, which are then contained in the finished pharmaceutical formulation, are pharmacologically compatible with the above-mentioned acid.

The formulations according to the invention may contain complexing agents as further ingredients. By complexing agents are meant within the scope of the present invention molecules which are capable of entering into complex bonds. Preferably, these compounds should have the effect of complexing cations, most preferably metal cations. The formulations according to the invention preferably contain editic acid (EDTA) or one of the known salts thereof, e.g. sodium EDTA or disodium EDTA, as complexing agent. Preferably, disodium edetate is used, optionally in the form of its hydrates, more preferably in the form of its dihydrate.

If complexing agents are used within the scope of the formulations according to the invention, their content is preferably in the range from 1 to 50 mg per 100 ml, particularly preferably in the range from 2 to 15 mg per 100 ml of the formulation according to the invention. Preferably the formulations according to the invention contain a complexing agent in an amount of about 4 to 12 mg per 100 ml, particularly preferably about 10 mg per 100 ml of the formulation according to the invention.

The remarks made concerning disodium edetate also apply analogously to other possible additives which are comparable to EDTA or the salts thereof, which have complexing properties and can be used instead of them, such as for example nitrilotriacetic acid and the salts thereof.

Other pharmacologically acceptable excipients may also be added to the formulation according to the invention. By adjuvants and additives are meant, in this context, any pharmacologically acceptable and therapeutically useful substance which is not an active substance, but can be formulated together with the active substance in the pharmacologically suitable solvent, in order to improve the qualitative properties of the active substance formulation. Preferably, these substances have no pharmacological effects or no appreciable or at least no undesirable pharmacological effects in the context of the desired therapy. The adjuvants and additives include, for example, stabilisers, antioxidants and/or preservatives which prolong the shelf life of the finished pharmaceutical formulation, as well as flavourings, vitamins and/or other additives known in the art. The additives also include pharmacologically acceptable salts such as sodium chloride, for example.

The preferred excipients include antioxidants such as ascorbic acid, for example, provided that it has not already been used to adjust the pH, vitamin A, vitamin E, tocopherols and similar vitamins or provitamins occurring in the human body.

Preservatives can be added to protect the formulation from contamination with pathogenic bacteria. Suitable preservatives are those known from the prior art, particularly benzalkonium chloride in the concentrations known from the prior art. Preferably, benzalkonium chloride is added to the formulation according to the invention. The amount of benzalkonium chloride added is between 1 mg and 50 mg per 100 ml formulation, preferably about 2 to 15 mg per 100 ml, particularly preferably about 3 to 12 mg per 100 ml, particularly preferably about 4 to 10 mg per 100 ml of the formulation according to the invention. Benzalkonium chloride may also be used according to the invention in admixture with other preservatives.

Preferred formulations contain only benzalkonium chloride, sodium edetate and the acid needed to adjust the pH, besides the solvent water and ethanol and the compounds of formula 1.

The medicament formulations according to the invention with compounds of formula 1 are preferably used in an inhaler of the type described hereinbefore in order to produce the propellant-free aerosols according to the invention. Specific mention should therefore be made once again of the patent documents described hereinbefore to which reference is hereby made.

As described at the beginning, a further developed embodiment of the preferred inhaler is disclosed in WO 97/12687 (cf. in particular FIGS. 6a and 6b and the associated passages of description). This nebuliser (Respimat®) can advantageously be used to produce the inhalable aerosols according to the invention. Because of its cylindrical shape and handy size of less than 9 to 15 cm long and 2 to 4 cm wide, the device can be carried anywhere by the patient. The nebuliser sprays a defined volume of the pharmaceutical formulation out through small nozzles at high pressures, so as to produce inhalable aerosols.

The preferred atomiser essentially consists of an upper housing part, a pump housing, a nozzle, a locking clamp, a spring housing, a spring and a storage container, characterised by
a pump housing fixed in the upper housing part and carrying at one end a nozzle body with the nozzle or nozzle arrangement,
a hollow piston with valve body,
a power take-off flange in which the hollow body is fixed and which is located in the upper housing part,
a locking clamping mechanism located in the upper housing part,
a spring housing with the spring located therein, which is rotatably mounted on the upper housing part by means of a rotary bearing,
a lower housing part which is fitted onto the spring housing in the axial direction.

The hollow piston with valve body corresponds to a device disclosed in WO 97/12687. It projects partially into the cylinder of the pump housing and is disposed to be axially movable in the cylinder. Reference is made particularly to FIGS. 1-4—especially FIG. 3—and the associated parts of the description of the above-mentioned International Patent Application. At the moment of release of the spring the hollow piston with valve body exerts, at its high pressure end, a pressure of 5 to 60 Mpa (about 50 to 600 bar), preferably 10 to 60 Mpa (about 100 to 600 bar) on the fluid, the measured amount of active substance solution. Volumes of 10 to 50 microlitres are preferred, volumes of 10 to 20 microlitres are more preferable, whilst a volume of 10 to 15 microlitres per actuation is particularly preferred.

The valve body is preferably mounted at the end of the hollow piston which faces the nozzle body.

The nozzle in the nozzle body is preferably microstructured, i.e. Manufactured by micro-engineering. Microstructured nozzle bodies are disclosed for example in WO 99/16530; reference is hereby made to the contents thereof, especially FIG. 1 and the associated description. The nozzle body consists for example of two sheets of glass and/or silicon securely fixed together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns and the length being 7 to 9 microns. If there is a plurality of nozzle openings, preferably two, the directions of spraying of the nozzles in the nozzle body may run parallel to each other or may be inclined relative to one another in the direction of the nozzle opening. In the case of a nozzle body having at least two nozzle openings at the outlet end, the directions of spraying may be inclined relative to one another at an angle of 20 degrees to 160 degrees, preferably at an angle of 60 to 150 degrees, most preferably 80 to 100°.

The nozzle openings are preferably arranged at a spacing of 10 to 200 microns, more preferably at a spacing of 10 to 100 microns, still more preferably 30 to 70 microns. A spacing of 50 microns is most preferred. The directions of spraying therefore meet in the region of the nozzle openings.

As already mentioned, the liquid pharmaceutical preparation hits the nozzle body at an entry pressure of up to 600 bar, preferably 200 to 300 bar and is atomised through the nozzle openings into an inhalable aerosol. The preferred particle sizes of the aerosol are up to 20 microns, preferably 3 to 10 microns.

The locking clamping mechanism contains a spring, preferably a cylindrical helical compression spring as a store for the mechanical energy. The spring acts on the power take-off flange as a spring member the movement of which is determined by the position of a locking member. The travel of the power take-off flange is precisely limited by an upper stop and a lower stop. The spring is preferably tensioned via a stepping-up gear, e.g. a helical sliding gear, by an external torque which is generated when the upper housing part is turned relative to the spring housing in the lower housing part. In this case, the upper housing part and the power take-off flange contain a single- or multi-speed spline gear.

The locking member with the engaging locking surfaces is arranged in an annular configuration around the power take-off flange. It consists for example of a ring of plastics or metal which is inherently radially elastically deformable. The ring is arranged in a plane perpendicular to the axis of the atomiser. After the tensioning of the spring, the locking surfaces of the locking member slide into the path of the power take-off flange and prevent the spring from being released. The locking member is actuated by means of a button. The actuating button is connected or coupled to the locking member. In order to actuate the locking clamping mechanism the actuating button is moved parallel to the annular plane, preferably into the atomiser, and the deformable ring is thereby deformed in the annular plane. Details of the construction of the locking clamping mechanism are described in WO 97/20590.

The lower housing part is pushed axially over the spring housing and covers the bearing, the drive for the spindle and the storage container for the fluid.

When the atomiser is operated, the upper part of the housing is rotated relative to the lower part, the lower part taking the spring housing with it. The spring meanwhile is compressed and biased by means of the helical sliding gear, and the clamping mechanism engages automatically. The angle of rotation is preferably a whole-number fraction of 360 degrees, e.g. 180 degrees. At the same time as the spring is tensioned, the power take-off component in the upper housing part is moved along by a given amount, the hollow piston is pulled back inside the cylinder in the pump housing, as a result of which some of the fluid from the storage container is sucked into the high pressure chamber in front of the nozzle.

If desired, a plurality of replaceable storage containers containing the fluid to be atomised can be inserted in the atomiser one after another and then used. The storage container contains the aqueous aerosol preparation according to the invention.

The atomising process is initiated by gently pressing the actuating button. The clamping mechanism then opens the way for the power take-off component. The biased spring pushes the piston into the cylinder in the pump housing. The fluid emerges from the nozzle of the atomiser in the form of a spray.

Further details of the construction are disclosed in PCT applications WO 97/12683 and WO 97/20590, to which reference is hereby made.

The components of the atomiser (nebuliser) are made of a material suitable for their function. The housing of the atomiser and—if the function allows—other parts as well are preferably made of plastics, e.g. by injection moulding. For medical applications, physiologically acceptable materials are used.

FIGS. 6 a/b of WO 97/12687 show the nebuliser (Respimat®) with which the aqueous aerosol preparations according to the invention can advantageously be inhaled. FIG. 6 a shows a longitudinal section through the atomiser with the spring under tension, FIG. 6 b shows a longitudinal section through the atomiser with the spring released.

The upper housing part (51) contains the pump housing (52), on the end of which is mounted the holder (53) for the atomiser nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58). The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66).

The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomised. The storage container is closed off by the stopper (73), through which the hollow piston projects into the storage container and dips its end into the fluid (supply of active substance solution).

The spindle (74) for the mechanical counter is mounted on the outside of the spring housing. The drive pinion (75) is located at the end of the spindle facing the upper housing part. On the spindle is the slider (76).

The nebuliser described above is suitable for nebulising the aerosol preparations according to the invention to form an aerosol suitable for inhalation.

If the formulation according to the invention is nebulised using the method described above (Respimat®), the mass expelled, in at least 97%, preferably at least 98% of all the actuations of the inhaler (puff or puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

The formulation according to the invention can also be nebulised using inhalers other than those described above, for example jet-stream inhalers.

The present invention also relates to an inhalation kit consisting of one of the pharmaceutical preparations according to the invention described above and an inhaler suitable for nebulising this pharmaceutical preparation.

The present invention preferably relates to an inhalation kit consisting of one of the pharmaceutical preparations according to the invention described above and the Respimat® inhaler described above.

The formulation examples that follow serve to provide further illustration without restricting the subject-matter of the present invention to the compositions described by way of example.

### Experimental Section

The compounds according to the invention may be prepared analogously to the methods known from the prior art. Suitable preparation methods are known for example from EP 1562603 or U.S. Pat. No. 7,056,916, the contents of which are hereby included by reference.

### Formulation Examples

A) Examples of novel medicament formulations of the R-enantiomer of compound Example 1a*HCl contain
23.3, 24.8, 33.7, 38.4, 42.4, 46.1, 49.5, 53.8, 60.2 or 68.3 mg of the active substance, as well as 10 mg benzalkonium chloride, 10 mg disodium edetate, 3 mg anhydrous citric acid and are topped up to 100 ml with purified water or water for injections or
23.3, 24.8, 33.7, 38.4, 42.4, 46.1, 49.5, 53.8, 60.2 or 68.3 mg of the active substance, as well as 8 mg benzalkonium chloride, 8 mg disodium edetate, 4 mg anhydrous citric acid and are topped up to 100 ml with purified water or water for injections or
23.3, 24.8, 33.7, 38.4, 42.4, 46.1, 49.5, 53.8, 60.2 or 68.3 mg of the active substance, as well as 5 mg benzalkonium chloride, 15 mg disodium edetate, 2 mg anhydrous citric acid and are topped up to 100 ml with purified water or water for injections or
23.3, 24.8, 33.7, 38.4, 42.4, 46.1, 49.5, 53.8, 60.2 or 68.3 mg of the active substance, as well as 15 mg benzalkonium chloride, 10 mg disodium edetate, 2 mg anhydrous citric acid and are topped up to 100 ml with purified water or water for injections.
   B) Examples of novel medicament formulations of the R-enantiomer of compound Example 1b*HCl contain:
23.7, 24.8, 33.7, 38.4, 42.4, 46.1, 49.5, 53.8, 60.2 or 68.3 mg of the active substance, as well as 10 mg benzalkonium chloride, 10 mg disodium edetate, 3 mg anhydrous citric acid and are topped up to 100 ml with purified water or water for injections or
23.7, 24.8, 33.7, 38.4, 42.4, 46.1, 49.5, 53.8, 60.2 or 68.3 mg of the active substance, as well as 8 mg benzalkonium chloride, 8 mg disodium edetate, 4 mg anhydrous citric acid and are topped up to 100 ml with purified water or water for injections or
23.7, 24.8, 33.7, 38.4, 42.4, 46.1, 49.5, 53.8, 60.2 or 68.3 mg of the active substance, as well as 5 mg benzalkonium chloride, 15 mg disodium edetate, 2 mg anhydrous citric acid and are topped up to 100 ml with purified water or water for injections or
23.7, 24.8, 33.7, 38.4, 42.4, 46.1, 49.5, 53.8, 60.2 or 68.3 mg of the active substance, as well as 15 mg benzalkonium chloride, 10 mg disodium edetate, 2 mg anhydrous citric acid and are topped up to 100 ml with purified water or water for injections.
   C) Examples of novel medicament formulations of the R-enantiomer of compound Example 1c*HCl contain:
23.6, 24.8, 33.7, 38.4, 42.4, 46.1, 49.5, 53.8, 60.2 or 68.3 mg of the active substance, as well as 10 mg benzalkonium chloride, 10 mg disodium edetate, 3 mg anhydrous citric acid and are topped up to 100 ml with purified water or water for injections or
23.6, 24.8, 33.7, 38.4, 42.4, 46.1, 49.5, 53.8, 60.2 or 68.3 mg of the active substance, as well as 8 mg benzalkonium chloride, 8 mg disodium edetate, 4 mg anhydrous citric acid and are topped up to 100 ml with purified water or water for injections or
23.6, 24.8, 33.7, 38.4, 42.4, 46.1, 49.5, 53.8, 60.2 or 68.3 mg of the active substance, as well as 5 mg benzalkonium chloride, 15 mg disodium edetate, 2 mg anhydrous citric acid and are topped up to 100 ml with purified water or water for injections or
23.6, 24.8, 33.7, 38.4, 42.4, 46.1, 49.5, 53.8, 60.2 or 68.3 mg of the active substance, as well as 15 mg benzalkonium chloride, 10 mg disodium edetate, 2 mg anhydrous citric acid and are topped up to 100 ml with purified water or water for injections.
   D) Examples of novel medicament formulations of the R-enantiomer of compound Example 1d*HCl contain:
24.1, 24.8, 33.7, 38.4, 42.4, 46.1, 49.5, 53.8, 60.2 or 68.3 mg of the active substance, as well as 10 mg benzalkonium chloride, 10 mg disodium edetate, 3 mg anhydrous citric acid and are topped up to 100 ml with purified water or water for injections or
24.1, 24.8, 33.7, 38.4, 42.4, 46.1, 49.5, 53.8, 60.2 or 68.3 mg of the active substance, as well as 8 mg benzalkonium chloride, 8 mg disodium edetate, 4 mg anhydrous citric acid and are topped up to 100 ml with purified water or water for injections or
24.1, 24.8, 33.7, 38.4, 42.4, 46.1, 49.5, 53.8, 60.2 or 68.3 mg of the active substance, as well as 5 mg benzalkonium chloride, 15 mg disodium edetate, 2 mg anhydrous citric acid and are topped up to 100 ml with purified water or water for injections or
24.1, 24.8, 33.7, 38.4, 42.4, 46.1, 49.5, 53.8, 60.2 or 68.3 mg of the active substance, as well as 15 mg benzalkonium chloride, 10 mg disodium edetate, 2 mg anhydrous citric acid and are topped up to 100 ml with purified water or water for injections.
   E) Examples of novel medicament formulations of the R-enantiomer of compound Example 1e*HCl contain:
23.8, 24.8, 33.7, 38.4, 42.4, 46.1, 49.5, 53.8, 60.2 or 68.3 mg of the active substance, as well as 10 mg benzalkonium chloride, 10 mg disodium edetate, 3 mg anhydrous citric acid and are topped up to 100 ml with purified water or water for injections or
23.8, 24.8, 33.7, 38.4, 42.4, 46.1, 49.5, 53.8, 60.2 or 68.3 mg of the active substance, as well as 8 mg benzalkonium chloride, 8 mg disodium edetate, 4 mg anhydrous citric acid and are topped up to 100 ml with purified water or water for injections or
23.8, 24.8, 33.7, 38.4, 42.4, 46.1, 49.5, 53.8, 60.2 or 68.3 mg of the active substance, as well as 5 mg benzalkonium chloride, 15 mg disodium edetate, 2 mg anhydrous citric acid and are topped up to 100 ml with purified water or water for injections or
23.8, 24.8, 33.7, 38.4, 42.4, 46.1, 49.5, 53.8, 60.2 or 68.3 mg of the active substance, as well as 15 mg benzalkonium chloride, 10 mg disodium edetate, 2 mg anhydrous citric acid and are topped up to 100 ml with purified water or water for injections.

1. Medicament formulation, comprising
as sole active substance one or more compounds of general formula 1 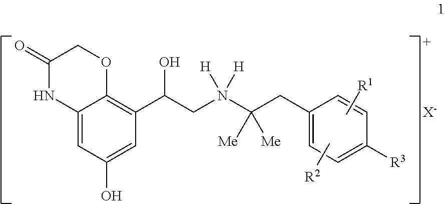
wherein
R¹ denotes hydrogen, fluorine, chlorine or methyl;
R² denotes hydrogen, fluorine, chlorine or methyl;
R³ denotes methyl, methoxy, ethoxy, fluorine, chlorine, bromine, O—CH₂—COOH or O—CH₂—COOethyl;
X⁻ denotes an anion with a single negative charge selected from among chloride, bromide, sulphate, methanesulphonate, maleate, acetate, benzoate, citrate, salicylate, trifluoroacetate, fumarate, tartrate and succinate;
optionally in the form of the tautomers, enantiomers, mixtures of enantiomers, racemates or solvates thereof;
benzalkonium chloride;
at least one pharmacologically acceptable acid,
and, as solvent, water, ethanol or a mixture of water and ethanol,
optionally further pharmacologically acceptable excipients and/or complexing agents
wherein the content of compound 1 is 55 to 200 μmol per 100 ml solution.

2. Medicament formulation according to claim 1, wherein said formulation comprises one or more compounds of formula 1, wherein
R³ denotes methoxy, ethoxy, fluorine, O—CH₂—COOH, O—CH₂—COOmethyl or O—CH₂—COOethyl;
optionally in the form of the tautomers, enantiomers, mixtures of enantiomers, racemates or solvates thereof.

3. Medicament formulation according to claim 1, wherein said formulation comprises one or more compounds of formula 1, wherein
R¹ and R² denotes hydrogen or fluorine;
R³ denotes fluorine, methoxy, ethoxy, O—CH₂—COOH;
optionally in the form of the tautomers, enantiomers, mixtures of enantiomers, racemates or solvates thereof.

4. Medicament formulation according to claim 1, wherein the pharmacologically acceptable acid is selected from among the inorganic acids hydrochloric acid, hydrobromic acid, nitric acid, sulphuric acid and phosphoric acid or from the organic acids ascorbic acid, citric acid, malic acid, tartaric acid, maleic acid, succinic acid, fumaric acid, acetic acid, formic acid and propionic acid.

5. Medicament formulation according to claim 1, wherein said formulation has a pH of 2.5 to 6.5.

6. Medicament formulation according to claim 1, wherein the content of benzalkonium chloride is 1 to 50 mg per 100 ml solution.

7. Medicament formulation according to claim 1, wherein said formulation comprises a complexing agent as a further ingredient.

8. Medicament formulation according to claim 7, wherein the content of complexing agent is 1 to 50 mg per 100 ml solution.

9. Medicament formulation according to claim 1, wherein said formulation comprises pure water as solvent.

10. Medicament formulation according to claim 1, characterised in that it contains as solvent a mixture of water and ethanol in which the percentage mass of ethanol is in the range between 5 and 99% ethanol.

11. Method for the treatment of respiratory complaints comprising administering to a patient a therapeutically effective amount of a formulation according to claim 1.

12. Inhalation kit consisting of a medicament formulation according to claim 1 and an inhaler suitable for nebulising this medicament formulation.

13. Inhalation kit according to claim 12, wherein the inhaler is the Respimat®.
